(19) United States
(12) Patent Application Publication
(10) Pub. No.: US 2015/0112228 A1
(43) Pub. Date: Apr. 23, 2015

(54) Bagless, dry, odorless catheter

(71) Applicants: George Mbella Ekema, Akron, OH (US); Marjet Danteel Heitzer, Akron, OH (US)

(72) Inventors: George Mbella Ekema, Akron, OH (US); Marjet Danteel Heitzer, Akron, OH (US)

(21) Appl. No.: 14/515,645

(22) Filed: Oct. 16, 2014

Related U.S. Application Data
(60) Provisional application No. 61/893,986, filed on Oct. 22, 2013.

(51) Int. Cl. A61F 5/44; A61F 13/49; A61B 5/1477; A61F 5/441; A61B 10/00; A61B 5/145

(52) U.S. Cl. CPC A61F 5/4408; A61B 10/007; A61B 5/14507; A61B 5/1477; A61F 5/441; A61F 13/49; A61F 5/4401
USPC 600/584; 604/385.03; 604/318; 604/544

(57) ABSTRACT

A bagless catheter and incontinence device, which reduces maceration, microbial skin infiltration and infection, urinary tract infection, and offers the user a normal, odor-free, active lifestyle, is provided.

### BACKGROUND OF THE INVENTION

1. Field of the Invention

The present invention relates to catheter and incontinence devices and, more particularly, to internal and external catheter devices configured for the human male and female anatomy.

2. Brief Discussion of the Related Art

Urinary incontinence, a common medical problem, is currently treated with external catheters, invasive catheters, incontinence pads and diapers.

There are several problems associated with the current treatments, most prominent of which is a high incidence of infections and preclusion from an active lifestyle. The incidence of urinary tract infections with indwelling invasive urinary catheters is well described in current medical literature. The presence of an invasive catheter in the urethra is thought to facilitate the infiltration of microorganisms. Microorganisms at the catheter entrance site ascend the catheter through the urethra into the urinary tract, thus resulting in infections. The conventional condom catheter is essentially a bag connected to a tube. This sheath is usually attached to the penis by a circumferential adhesive band. Often the attachment is inadequate, and the condom catheter falls off or leaks. In other cases, the circumferential adhesive band results in irritation or even strangulation of the penis. The condom catheter design presents no barrier to the infiltration of microorganisms into the urethra. Inside the bag, the moist environment is ideal for microorganism growth.

Microorganisms growing in the moist environment in the bag can enter the urethra and proceed into the urinary tract, causing urinary tract infections. Incontinence pads or diapers, whether used on male or female patients, also contribute to the onset and severity of infections because the excreted urine and/or feces contained in these devices are excellent media for bacterial growth. Such bacteria can enter the urethra because in long-term care facilities personnel are often unable to change patients' diapers as frequently as recommended by good medical practice. The second problem associated with current incontinence treatments is particularly related to the condom catheter. The conventional condom catheter causes skin maceration and irritation. The epidermis is composed of dead cells, which protect the underlying living dermis cells from physical and chemical irritation and from microbial invasion. Maceration refers to the problems associated with prolonged exposure of the epidermis to moisture. When epidermal cells are exposed to moisture for a sufficient period of time, they absorb water, swell, and slough off, thus exposing the physiologically active dermal layer to whatever chemical or microbial entities are present in the immediate environment. Maceration occurs not only when the skin is exposed to water from external sources but also when moisture is trapped against the skin surface by a water-impermeable membrane. Skin continually emits water, particularly in the genital area. If the emitted water is trapped against the skin surface by a water-impermeable membrane and is not able to evaporate, then maceration occurs.

If maceration proceeds unabated, an inflammation of cellular and connective tissue caused by the failure of the epidermis, often occurs. This inflammation is often accompanied or followed by microbial invasion, causing cellulitis or tissue infection. Microbial invasion causes further tissue inflammation and destruction. In severe cases of cellulitis, extensive tissue destruction, necrosis or gangrene ensues, necessitating removal of the gangrenous tissues or the entire affected limb. Clearly, maintaining skin dryness is necessary to prevent the onset of maceration and further degenerative states.

The conventional condom catheter functions as a bag of moisture surrounding the penis. The condom catheter does not allow the necessary evaporation of moisture from the skin surface. Not only do the walls of the condom trap the emitted moisture against the skin surface, but urination also adds to the moisture trapped inside the condom and contributes chemical irritants, such as urea, to the enclosed area. Thus, conventional condom catheters cause maceration and skin irritation.

The maceration and skin irritation caused by condom catheters make them physically uncomfortable, and patients routinely remove them, either deliberately or inadvertently while sleeping. In addition, macerated tissue is moist and loose. Adhesive, therefore, does not adhere well to macerated skin. Thus, adding a simple adhesive to the condom catheter does not help, because the condom catheter becomes loose or falls off of the patient due to maceration under the adhesive.

The conventional urinary catheter comprises a bag or bed pan for the collection of urine. Carrying along a bag of swooshing urine precludes the user from engaging, normally, in an active lifestyle. A bag of urine also causes odor.

In connection with a male external urinal device of the type using a urine receptacle worn on the body or near the body, it is common to use a sheath of flexible material placed over the penis and connected to the receptacle with a tube or other form of flexible conduit. Some devices customarily include a narrow tape wound relatively tightly about the sheath to hold the sheath securely and prevent leakage. Since urinal devices must be worn for long periods of time, particularly in the case of paraplegics, wound tape exerts continuous pressure on a penis and particularly on the urethral passage, which is located on the under side of the penis fairly close to the surface. Constrictions and discomfort may result. It is not uncommon for a tightly wound tape to cause swelling or even gangrene. On the other hand, a loose tape can allow leakage of urine and resultant irritation.

In U.S. Pat. No. 3,863,638, Rogers shows a liner pad between the sheath and the penis to form a cushion and absorb some of the pressure exerted by an externally wound tape. Often, however, the pad is inadvertently not used by an attendant applying the device to a patient, or the pad is simply too wide or too narrow for a particular penis. Additionally, Rogers indicates the use of adhesive on both sides of the pad, thus eliminating the need for an external tape. Additionally, the device does not address dryness of the penis; neither does it take care of urine odor.

In U.S. Pat. No. 4,187,851, Hauser eliminates an external tape by showing the use of an adhesive on both the inner and outer surfaces of a pad. In this way, the pad can be wound around a penis, and the sheath of the urinal device rolled over the pad and held in place by the adhesive. Although the devices of Rogers and Hauser eliminate the externally wound tape, they continue to present the problems, which result whenever a material is too tightly wound about a penis. Additionally, the device does not address dryness of the penis; neither does it take care of urine odor.

Broerman in U.S. Pat. No. 3,739,783 shows a urinal device to be used without wound tape or pads. An adhesive is painted onto a penis, and a sheath impregnated with silicone rubber placed over the adhesive. The method and device, however, are not satisfactory since reliability continues to depend on an attendant's use of a proper adhesive and proper application of the adhesive for sealing the device. Additionally, the device does not address dryness of the penis; neither does it take care of urine odor.

U.S. RE33206 E teaches of a urinal device, including a laminated sheath having an inner layer of latex rubber and outer layer of silicone rubber. Adhesive is stored between the inner and outer layers when sheath is rolled. As sheath is unrolled, adhesive is released from outer layer and adheres to inner layer. Upon pressing sheath to a penis, a leak-free bond is created. The method and device are not satisfactory because of the dependence upon adhesive for a water-tight seal. Additionally, the device does not address dryness of the penis; neither does it take care of urine odor.

Hence, there continues to be a need for a safe, leak-free male urinal device.

In connection with a female external device, several patents teach of devices that are plagued with problems.

In U.S. Pat. No. 6,428,521 B1, Droll teaches of a female external device with a ladle-like geometry in which the spoon thereof is an intra-labial collection cup, and the handle is a hollow neck and knob that is secured within the vagina. Apertures with the knob enable venting of vaginal fluids through a fluid egress channel. This device is cumbersome and rigid. Additionally, it does not address dryness of the pubic region; neither does it take care of urine odor.

In U.S. Pat. No. 4,233,978 A, Hickey teaches of an external female catheter with an extremely thin wall that is of substantially flat configuration and completely covers the entire vulva area of the female. The extremely thin walls expand for temporary surges of expelled urine that increase the volume, but the thin walls return to the substantially flat configuration as the urine is drained out of the device. The device is reinforced at the edges by a thickening of the thin walls. The device is attached to the patient with an adhesive. While the thin wall is comparable to that of the instant device, Hickey's device has several problems. Firstly, the device lacks scaffolding and will not expand when urine is introduced if the user is wearing underwear or is in any position other than standing. Secondly, the adhesive does not allow for breathing of the device. Thirdly, the device does not address dryness of the pubic region; neither does it take care of urine odor.

In U.S. Pat. No. 4,484,917 A, Balckmon teaches of a female external device constructed from four major elements, an adhesive surface, a pliable stabilizer, a main body and an outlet tube. The adhesive surface covers the subject side of the stabilizer and surrounds the inlet to the main body of the catheter. The stabilizer is formed of a material conformable to the subject and aids in placing the catheter. Although the stabilizer can function as scaffolding for maintaining a urine-receiving pocket, the device still has significant problems. Firstly, the device lacks proper scaffolding, and the stabilizer is cumbersome and uncomfortable. Secondly, the adhesive does not allow for breathing of the device. Thirdly, the device does not address dryness of the pubic region; neither does it take care of urine odor.

In US 2011,0,028,982 A1, Kay et al. teach of a female external incontinence device including a housing formed of a soft, pliable, membranous, elastomeric material defining a flange for adhesive securement to the intralabial parameatal surface to create a parameatal seal around the urethral meatus, a urine collection channel and an outlet tube and an applicator having a tray component for holding the housing to facilitate securement of the housing to the intralabial parameatal surface. There are several significant problems with the device. Firstly, it requires a great deal of dexterity to attach the device to the intralabial parameatal surface. Secondly, the intralabial parameatal surface is relatively more sensitive to abrasion and maceration than the mons pubis and labia majora. Thirdly, the device does not address dryness of the pubic region; neither does it take care of urine odor.

In connection with internal catheter devices, many have been described in the prior art. They, however, have a common denominator of susceptibility to infections. Regardless of their design, they are susceptible to infection primarily because they are wettable. The instant device improves on the prior art by coating both the internal and external aspects of prior art devices with a super-hydrophobic layer—said super-hydrophobic layer conferring not-wettability to the devices and, therefore, rendering their surfaces unattachable to infection-causing microbes.

In connection with tubes, the wettability of prior art tubes causes or contributes to infections. The wettability of prior art tubes also contributes to urine odor. The instant device improves on the prior art tubes by coating the internal aspects of prior art tubes with a super-hydrophobic layer—said super-hydrophobic layer conferring not-wettability to the internal tube surface and, therefore, rendering the surfaces unattachable to infection-causing microbes. The not wettable surfaces also move urine more efficiently with none retained by the tube surface and consequently an odor-free tube.

In connection with the urine collection device, several devices using bags or containers have been taught in the prior art. The problem with the prior art devices is that being attached to a bag (or container) of urine precludes the attached individual from a normal, active lifestyle. It is also impossible to eliminate the odor of urine with the bags and containers taught in the prior art. The instant device eliminates bags and containers and allows the user to engage in a normal, odor-free, active lifestyle.

### SUMMARY OF THE INVENTION

It is therefore an objective of the present invention to reduce or eliminate microbial infiltration and the resulting infections and odor associated with the use of conventional urinary catheters, incontinence pads and diapers, while providing the user the ability to engage in a normal, odor-free, active lifestyle.

Yet, another objective of the present invention is to reduce or prevent maceration and skin irritation due to external catheter use.

An additional objective of the present invention is to provide a more comfortable external incontinence device, thus increasing patient compliance. Another objective of the present invention is to provide an external incontinence device that can be worn for several days or that can be disposed of daily.

Yet, an additional objective of the present invention is to create a dry catheter that eliminates the leaks and spills associated with urine collections bags, and that eliminates the handling of urine.

Yet, another objective of the present invention is to eliminate the unpleasant odor of urine that is characteristic of catheter and incontinence device use.

In satisfying these and other objectives, there has been provided, in accordance with one aspect of the present invention, a disposable, bagless urine collection device connected by at least one tube to a catheter device. The urine collection device comprises a bladder, with a free opening at its distal aspect, surrounded by at least one ultra-thin absorbent layer and a charcoal-plugged air valve in an air-tight, water-tight, pouch. Said air valve releasing gas from the sealed urine collection device by the pressure of the holster housing the device. The absorbent layer(s) absorb urine and prevent sloshing so that the user can freely engage in a relatively more active lifestyle.

In another aspect of the invention, the disposable, bagless urine collection device comprises a gauge that indicates the volume of urine absorbed and indicates when the device should be discarded.

In another aspect of the invention, the disposable, bagless urine collection device comprises a urinalysis strip.

In another aspect of the invention, the disposable, bagless urine collection device is connected by at least one tube to a male external catheter device. The male external catheter device comprises a thin condom-like pouch with two strips of vented, double-sided adhesive tape at its proximal two-thirds. The proximal two-thirds of the male external catheter device is separated from the distal one-third by a sealing membrane. In a preferred embodiment, the sealing membrane comprises two layers with drying agent (such as calcium chloride and magnesium sulfate) and charcoal at the peripheral (circumferential) aspect, and a slit at its center.

In another aspect of the invention, the disposable, bagless urine collection device is connected by at least one tube to a female external catheter device. The female external device is comprised of a receptacle, said receptacle attaches to the urethral opening area by a ventilated adhesive sheet that attaches to the mons pubis, lateral aspects to the labia majora, and perineum. The tubes attach at the inferior aspect of the receptacle. In a preferred embodiment, the tubes attach to the side (left or right) of the inferior aspect of the receptacle.

In another aspect of the invention, the interior of the bladder of the bagless urine collection device, the interior of the connecting tube(s), the interior of the male external catheter device, and the interior of the female external catheter device are coated with a super-hydrophobic layer.

In another aspect of the invention, the bagless urine collection device, the connecting tube(s), and the external male catheter device are a disposable unit.

In another aspect of the invention, the bagless urine collection device, the connecting tube(s), and the external male catheter device are a disposable unit comprising of biodegradable plastic and rubber.

In another aspect of the invention, the bagless urine collection device, the connecting tube(s), and the external female catheter device are a disposable unit.

In another aspect of the invention, the bagless urine collection device, the connecting tube(s), and the external female catheter device are a disposable unit comprising of biodegradable plastic and rubber.

In another aspect of the invention, the disposable, bagless urine collection device is connected by at least one tube to a long-term internal catheter.

In another aspect of the invention, the internal and external surfaces of the internal catheter are coated with a super-hydrophobic layer.

In another aspect of the invention, the bagless urine collection device is contained in a holster. The holster holds the bagless urine collection device securely to the leg of the user. The holster comprises elastic aspects that exert pressure on the bagless urine collection device so as to automatically expel developing gas through the air valve of the device.

### BRIEF DESCRIPTION OF THE DRAWINGS

In order that the invention may be more readily understood, and so that further features thereof may be appreciated, the invention will now be described by way of example with reference to the accompanying drawings, in which FIGS. 1-5 relate to the holster and urine collection device, FIGS. 6-7 relate to the urine collection device coupled by tubes to the male external catheter device, FIGS. 8-11 relate to the male external catheter device, FIG. 12 relates to the female external catheter device, FIG. 13 relates to the female external catheter device coupled to the urine collection device by tubes, FIG. 14 relates to the spine of the female external catheter device, and FIG. 15 relates to the lip of the female external catheter device, and in which:

FIG. 1 is an external view of the holster.

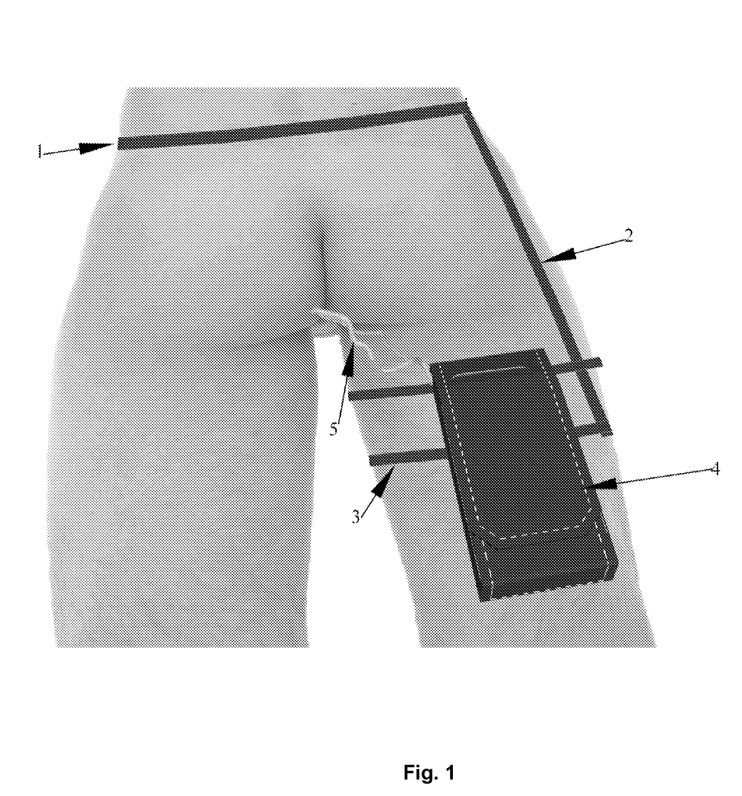

FIG. 2 is an x-ray view of the holster, showing the urine collection device and its bladder.

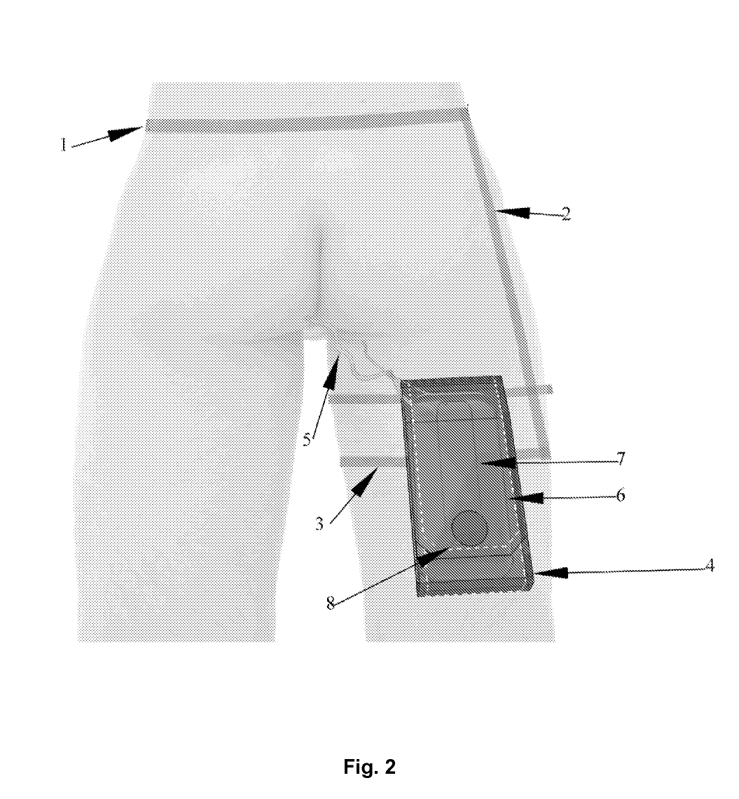

FIG. 3 is a side view of the external aspect of the holster.

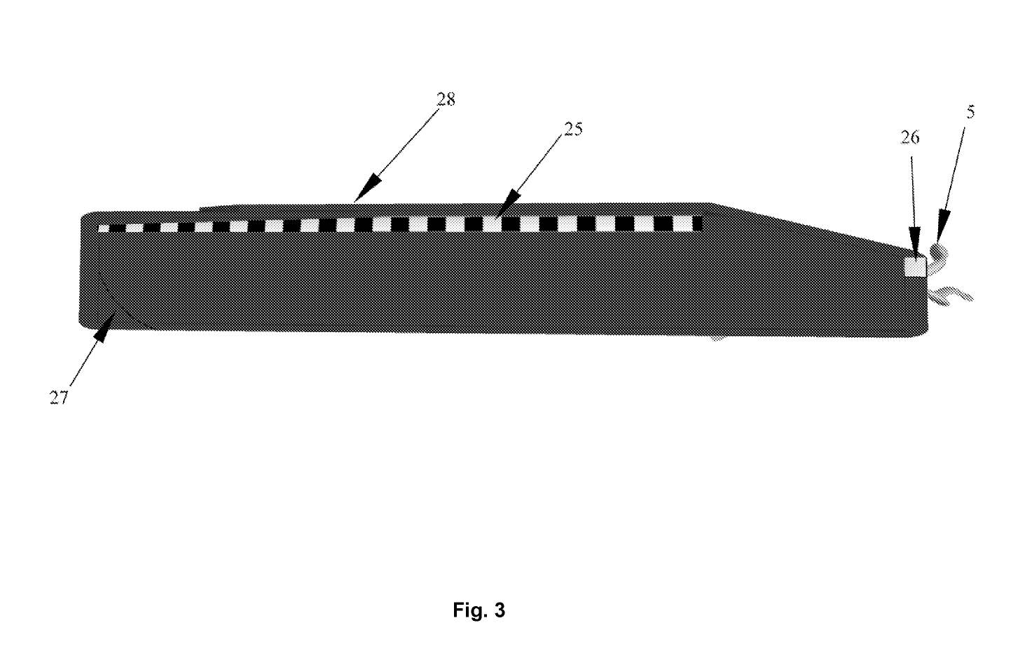

FIG. 4 is an x-ray side view of the holster, showing the urine collection device and its bladder.

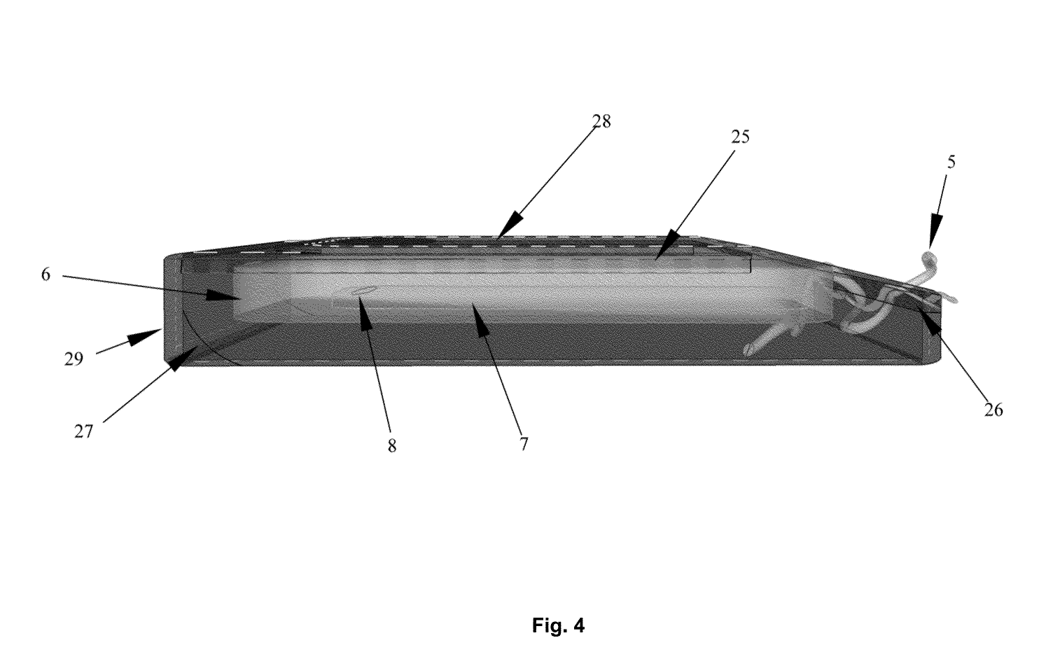

FIG. 5 is an x-ray superior view of the holster, showing the urine collection device and its bladder.

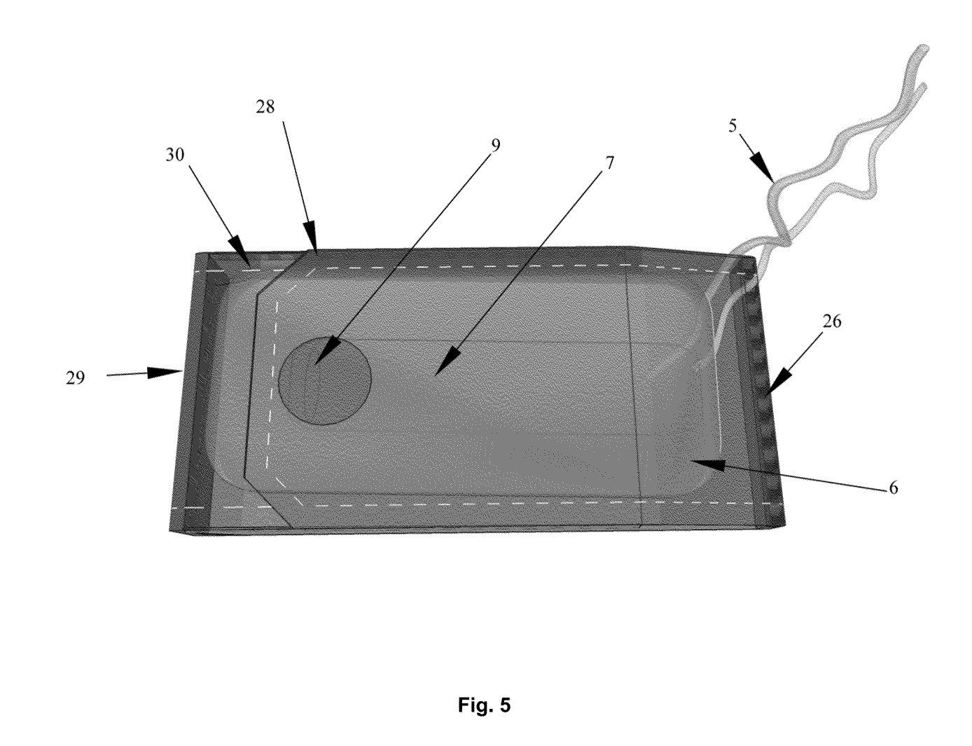

FIG. 6 is an x-ray view of the urine collection device coupled to the male external catheter device.

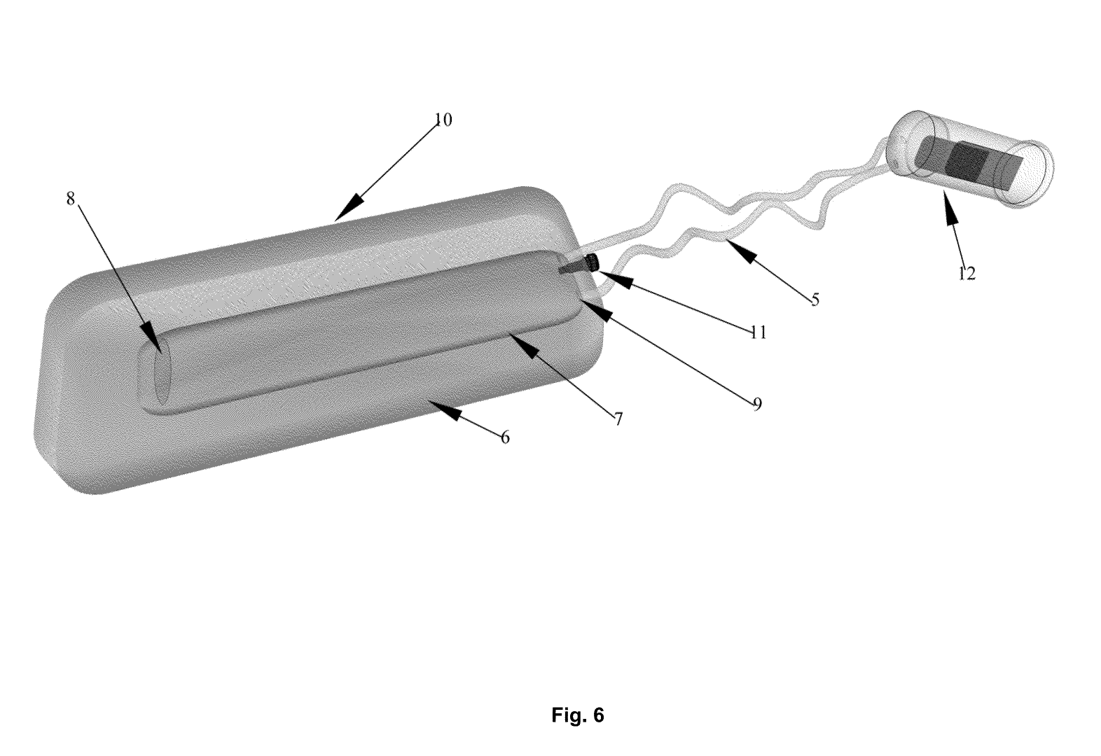

FIG. 7 is an x-ray view of the urine collection device coupled to the male external catheter device and showing additional components.

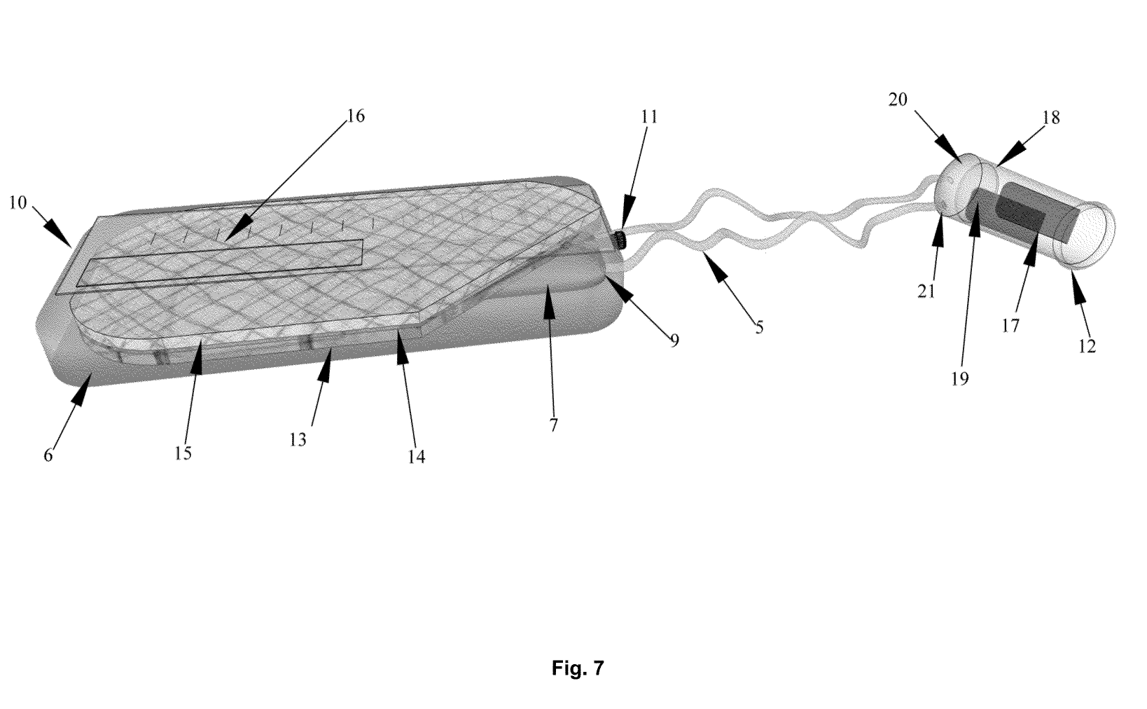

FIG. 8 is an x-ray view of the rolled-out male external catheter device.

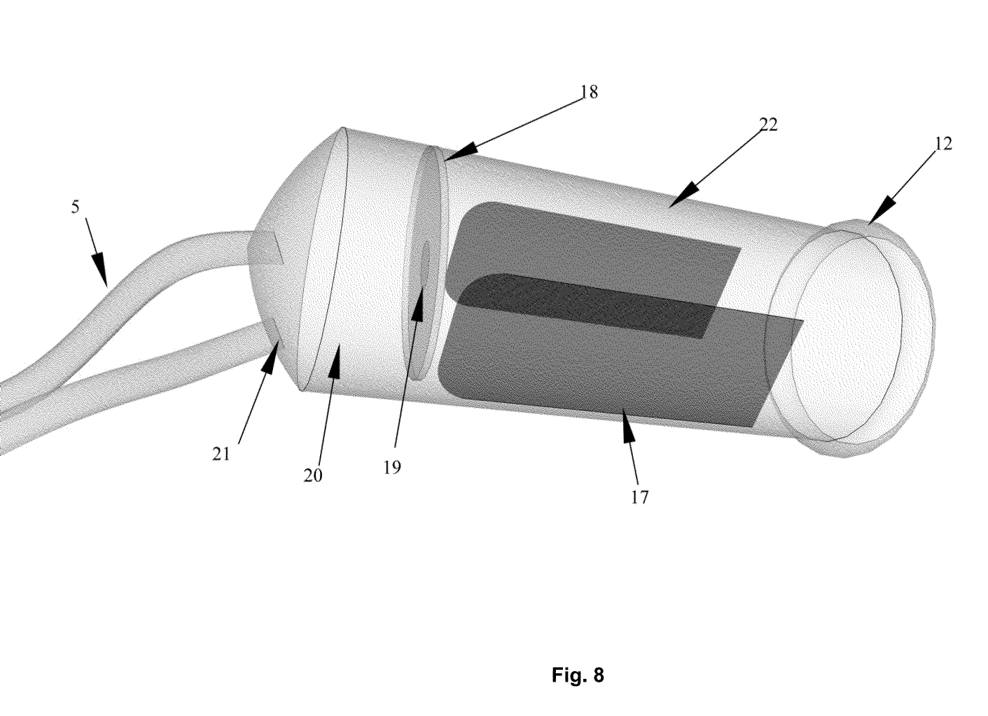

FIG. 9 is an external side view of the rolled male external catheter device.

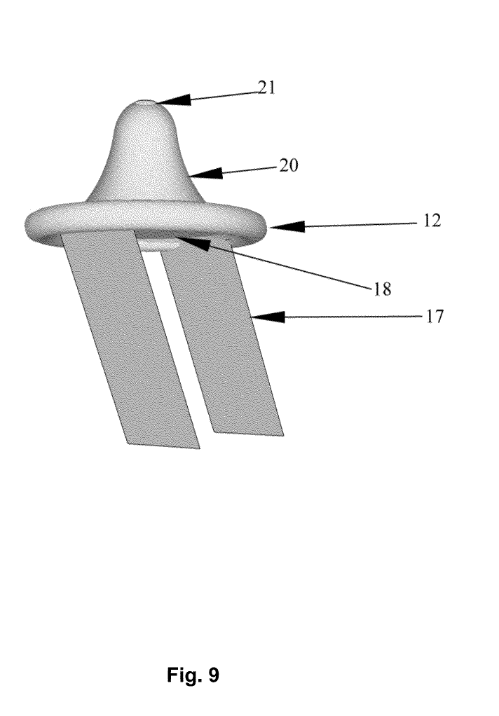

FIG. 10 is an external inferior view of the rolled male external catheter device.

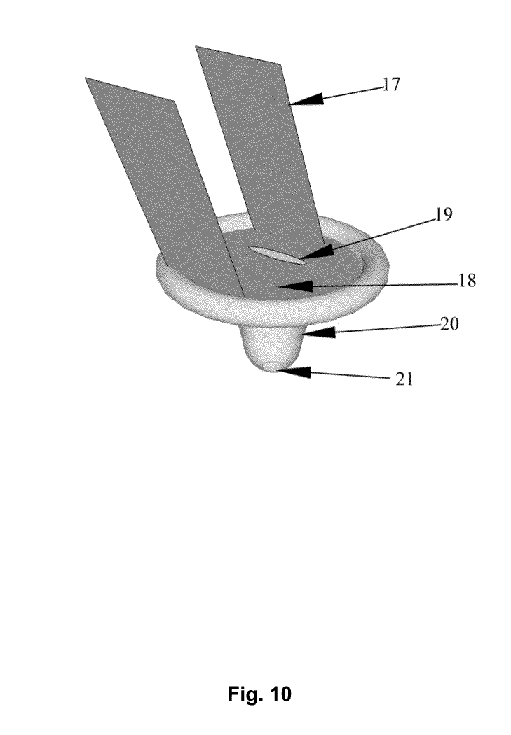

FIG. 11 is an external inferior/side view of the rolled male external catheter device.

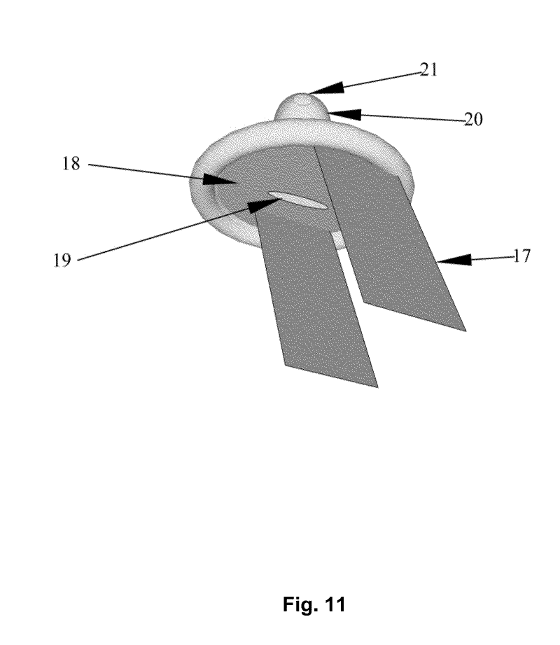

FIG. 12 is an external view of the female external catheter device.

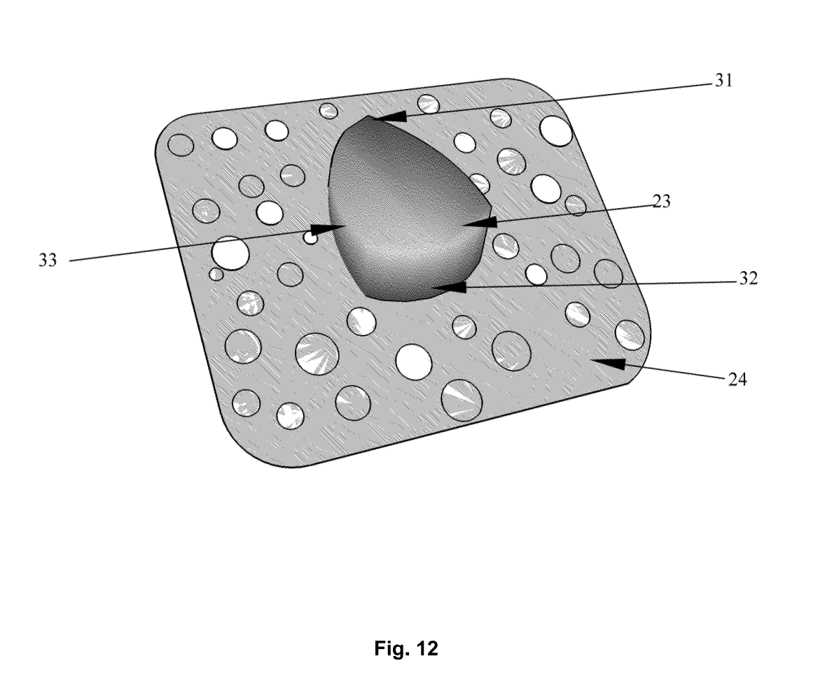

FIG. 13 is an x-ray view of the urine collection device coupled to the female external catheter device.

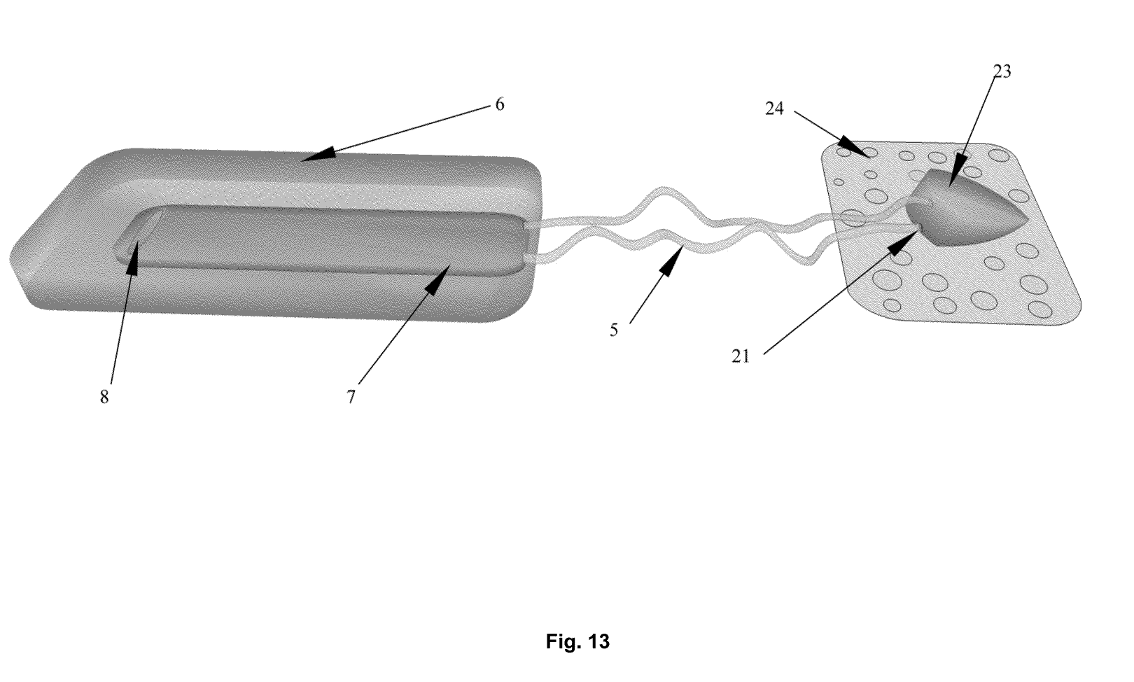

FIG. 14 is an x-ray view of the female external catheter device, showing the supporting spine.

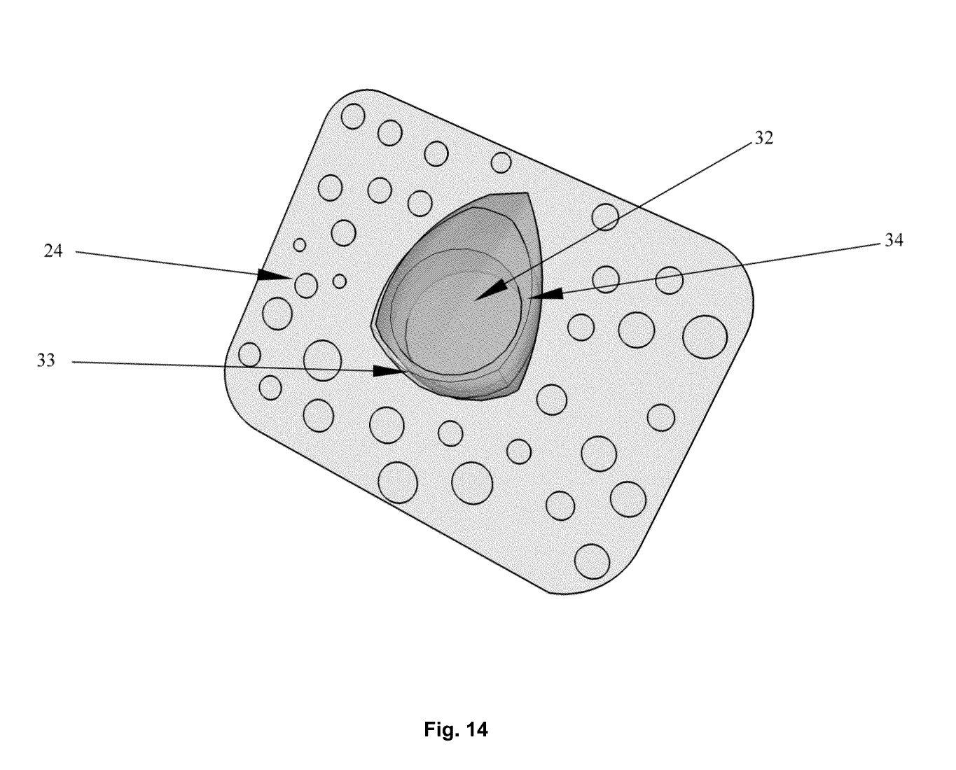

FIG. 15 is an inferior view of the female external catheter device showing the lip.

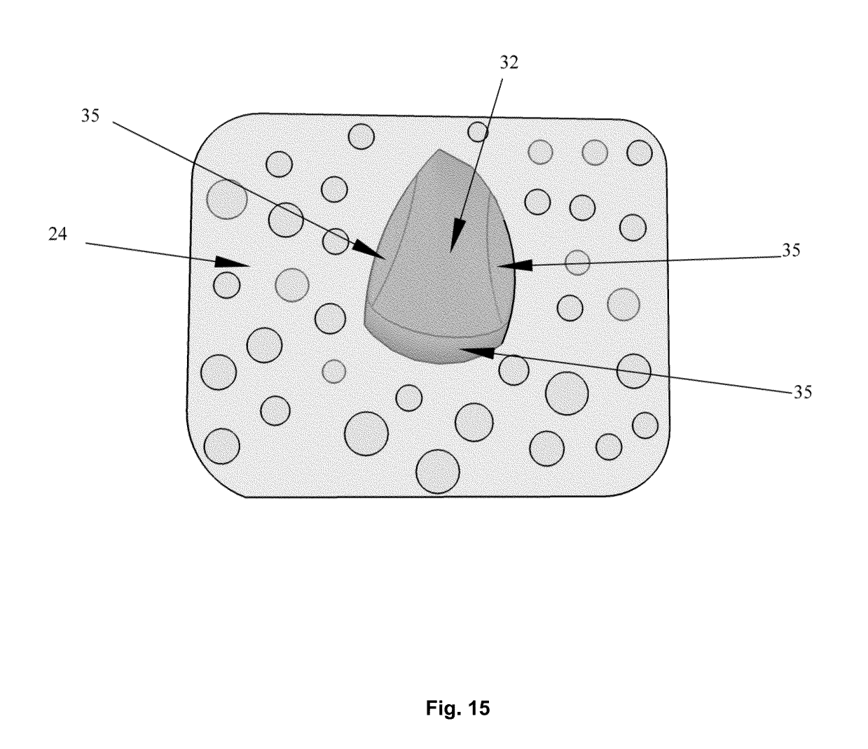

### DETAILED DESCRIPTION OF EXEMPLARY EMBODIMENTS OF THE INVENTION

In accordance with one aspect of the present invention, is a holster (4) suspended by a waist strap (1), vertical holster strap (2), and thigh strap (3). The holster (4) comprises a front elastic band (25) and a cover elastic band (26). The front elastic band (25) and cover elastic band (26) cause the holster cover (28) and front flap of the holster (30) to exert steady pressure on the urine collection device (6) to expel gas from the urine collection device (6) via the charcoal plug air valve (11). The holster (4) further comprises a bottom flap hinge (27) to allow for access through the inferior aspect of the holster (4) while the holster cover (28) allows for access through the superior aspect of the holster (4). In a preferred embodiment, the holster front flap (30) opens along one of its elastic bands (25) to allow for easy insertion and removal of the bagless urine collection device (6). Opening of the holster front flap (30) allows for easy usage by persons with limited dexterity.

Further in accordance with the objects of the invention, a bagless urine collection device (6) is provided. The bagless urine collection device (6) comprises a bladder (7) with a distal opening (8) at its inferior aspect and at least one proximal opening (9) connected to tube(s) (5) communicating with an external or internal catheter device. In a preferred embodiment, the interior aspect of the bladder (7) is coated with a super-hydrophobic layer; where the super-hydrophobic material is comprised of manganese oxide polystyrene (MnO₂/PS), zinc oxide polystyrene (ZnO/PS), precipitated calcium carbonate, carbon nano-tube structures, or silica nano-coating. In the preferred embodiment the interior aspect of the bladder (7) is coated with a silica-based super-hydrophobic layer. The bladder (7) communicates with an absorbent layer (13) such that the distal opening (8) drains its contents unto the absorbent layer (13). In the preferred embodiment, the absorbent layer (13) communicates with an ultra-thin absorbent layer (15) with a thin charcoal layer (14) between. The collection device is contained in an air-tight, water-tight, external cover (10). The external cover (10) is vented, at its superior aspect by a charcoal plug air valve (11) which allows for the automatic and immediate unidirectional evacuation of gas from the urine collection device (6).

The bagless urine collection device (6) further comprises of a gauge (16) that measures the volume of urine absorbed by the device and indicates if the device is approaching its capacity. In one embodiment, the gauge (16) comprises discrete strips of pH indicator paper. In the preferred embodiment, the gauge comprises discrete strips of wetness indicator material, including corn starch. In one embodiment, the level indicator is printed on the ultra-thin absorbent layer (15), which is then covered with discrete strips of wetness indicator material that reveals the indicator upon wetness. In yet another embodiment, the gauge (16) comprises a wetness indicator chemical, such as anhydrous copper sulfate that changes color upon hydration. In yet another embodiment, the outermost absorbent layer comprises a wetness indicator as taught in U.S. Pat. No. 6,075,178 A and herein incorporated by reference in its entirety. In yet another embodiment, the outermost absorbent layer comprises a wetness indicator as taught in WO 2001041691 A1 and herein incorporated by reference in its entirety. In yet another embodiment, the outermost absorbent layer comprises a wetness indicator for multiple fluid types as taught in US 20130066289 A1 and herein incorporated by reference in its entirety. In yet another embodiment, the outermost absorbent layer comprises a wetness indicator as taught in EP 1216673 B1 and herein incorporated by reference in its entirety. In yet another embodiment, the outermost absorbent layer comprises a wetness indicator as taught in US 20110137274 A1 and herein incorporated by reference in its entirety. In yet another embodiment, the outermost absorbent layer comprises a wetness indicator as taught in EP 1633296 B2 and herein incorporated by reference in its entirety. In yet another embodiment, the outermost absorbent layer comprises a wetness indicator as taught in U.S. Pat. No. 7,159,532 B2 and herein incorporated by reference in its entirety. In yet another embodiment, the outermost absorbent layer comprises a wetness indicator as taught in U.S. Pat. No. 4,231,370 A and herein incorporated by reference in its entirety. In yet another embodiment, the outermost absorbent layer comprises a wetness indicator as taught in U.S. Pat. No. 8,383,875 B2 and herein incorporated by reference in its entirety. In yet another embodiment, the outermost absorbent layer comprises a wetness indicator as taught in CA 1284002 C and herein incorporated by reference in its entirety. In yet another embodiment, the outermost absorbent layer comprises a wetness indicator as taught in US 20130138066 A1 and herein incorporated by reference in its entirety. In yet another embodiment, the outermost absorbent layer comprises a wetness indicator as taught in U.S. Pat. No. 5,342,861 A and herein incorporated by reference in its entirety.

In yet another embodiment, the urine collection device (6) comprises absorbent products and methods as described in EP 0,526,225 A1, EP 0,693,915 B1, EP 1,447,067 A1, U.S. Pat. No. 4,402,688, U.S. Pat. No. 8,075,538 B2, US 2006,0,079,854 A1, EP 0,391,814 A, U.S. Pat. No. 4,889,597 A, U.S. Pat. No. 4,935,022 A, each of which is hereby incorporated by reference in its entirety.

In yet another embodiment the urine collection device (6) is used as an ostomy device as described in U.S. Pat. No. 7,918,836 B2, U.S. Pat. No. 1,687,012 A, U.S. Pat. No. 2,310,505 A, U.S. Pat. No. 2,648,335 A, U.S. Pat. No. 2,887,109 A, U.S. Pat. No. 3,282,412 A, U.S. Pat. No. 3,292,626 A, U.S. Pat. No. 3,295,145 A, U.S. Pat. No. 3,297,152 A, U.S. Pat. No. 3,405,714 A, U.S. Pat. No. 3,559,651 A, U.S. Pat. No. 3,618,606 A, U.S. Pat. No. 3,780,739 A, U.S. Pat. No. 3,822,704 A, U.S. Pat. No. 3,902,496 A, U.S. Pat. No. 4,084,590 A, WO 9,427,530, each of which is hereby incorporated by reference in its entirety.

In one embodiment, the urine collection device (6) comprises a urinalysis strip. In a preferred embodiment, the urinalysis strip automatically assays the urine content in the urine collection device (6). In one embodiment, the urinalysis test strip comprises the methods taught in U.S. Pat. No. 8,217,217 B2 and herein incorporated by reference in its entirety. In one embodiment, the urinalysis test strip comprises the methods taught in U.S. Pat. No. 7,365,238 B2 and herein incorporated by reference in its entirety.

In one embodiment, the urine collection device (6) is connected to a male external catheter device (12) by at least one tube (5). The male external catheter device (12) is comprised of a condom-like tube with two strips of vented, double-sided adhesive tape (17) at its proximal two-thirds. The proximal two-thirds of the male external catheter device (12) is separated from the distal one-third by a sealing membrane (18). In the preferred embodiment, the sealing membrane (18) comprises two layers with drying agent (such as calcium chloride and magnesium sulfate) between the layers and charcoal at the peripheral (circumferential) aspect and a slit (19) at its center. The membrane slit seals the proximal aspect of the dry receptacle chamber (20) by sealing the penile shaft without applying so much pressure as to cause discomfort of strangulation. In a preferred embodiment, the interior aspects of the distal one-third of the male external catheter device (12) (internal walls of the dry receptacle chamber (20)) and the sealing membrane (18) are coated with a super-hydrophobic layer; where the super-hydrophobic material is comprised of manganese oxide polystyrene (MnO₂/PS), zinc oxide polystyrene (ZnO/PS), precipitated calcium carbonate, carbon nano-tube structures, or silica nano-coating. In the preferred embodiment, the interior aspects of the distal one-third of the male external catheter device (12) and the sealing membrane (18) are coated with a silica-based super-hydrophobic layer to form a dry receptacle chamber (20). The dry receptacle chamber (20) communicates with a tube (5) at its distal end. In the preferred embodiment, the dry receptacle chamber (20) communicates with more than one tube (5). In the most preferred embodiment, the dry receptacle chamber (20) communicates with two tubes (5). The interior aspects of the tubes (5) are coated with a super-hydrophobic layer where the super-hydrophobic material is comprised of manganese oxide polystyrene (MnO₂/PS), zinc oxide polystyrene (ZnO/PS), precipitated calcium carbonate, carbon nano-tube structures, or silica nano-coating. In the preferred embodiment, the interior aspects of the tube (5) are coated with a silica-based super-hydrophobic layer.

In another embodiment, the urine collection device (6) is connected to a female external catheter device (23) by at least one tube (5). The female external device comprises of a receptacle (32), said receptacle communicating at its inferior aspect with a tube (5). In the preferred embodiment, the inferior aspect of the female external device (32) communicates with more than one tube (5). In the most preferred embodiment, the inferior aspect of the female external device (32) communicates with two tubes (5). In a preferred embodiment, the interior aspects of the tubes (5) are coated with a super-hydrophobic layer where the super-hydrophobic material is comprised of manganese oxide polystyrene (MnO₂/PS), zinc oxide polystyrene (ZnO/PS), precipitated calcium carbonate, carbon nano-tube structures, or silica nano-coating. In the preferred embodiment, the interior aspects of the tube (5) are coated with a silica-based super-hydrophobic layer. In yet another embodiment, the external catheter devices (12 and 23) comprise latex, nitrile, polyurethane, polyisoprene, or lamb intestine. In yet another embodiment, the external catheter devices (12 and 23) comprise thin layers of latex, nitrile, polyurethane, polyisoprene, or lamb intestine and are disposable. In yet another embodiment, the external catheter devices (12 and 23) comprises products and methods as described in U.S. Pat. No. 5,263,947, U.S. Pat. No. 5,334,175, US 2005,0,177,133 A1, U.S. RE33206, U.S. Pat. No. 2,389,831, U.S. Pat. No. 3,403,682, U.S. Pat. No. 3,520,305, U.S. Pat. No. 3,739,783, U.S. Pat. No. 3,788,324, U.S. Pat. No. 3,835,857, U.S. Pat. No. 4,187,851, U.S. Pat. No. 4,284,079, U.S. Pat. No. 4,339,485, each of which is hereby incorporated by reference in its entirety.

In yet another embodiment, the female external catheter device (23) comprises a spine (34) that props the receptacle (32). In the preferred embodiment, the spine comprises a soft, malleable material such as packing peanuts and is coated with a super-hydrophobic layer where the super-hydrophobic material is comprised of manganese oxide polystyrene (MnO₂/PS), zinc oxide polystyrene (ZnO/PS), precipitated calcium carbonate, carbon nano-tube structures, or silica nano-coating. In the preferred embodiment, the internal and external aspects of the internal catheter device are coated with a silica-based super-hydrophobic layer. In the preferred embodiment, the spine (34) comprises charcoal and a drying agent, such as calcium chloride and magnesium sulfate.

In yet another embodiment, the female external catheter device (23) comprises a vaginal skin barrier for keeping the skin dry. The vaginal skin barrier comprises a urethral opening and is coated with a super-hydrophobic layer where the super-hydrophobic material is comprised of manganese oxide polystyrene (MnO₂/PS), zinc oxide polystyrene (ZnO/PS), precipitated calcium carbonate, carbon nano-tube structures, or silica nano-coating. In the preferred embodiment, the internal and external aspects of the internal catheter device are coated with a silica-based super-hydrophobic layer.

In yet another embodiment, the female external catheter device (23) comprises a lip (35) at its side and inferior aspects to prevent the flow of urine toward the skin. The lip (35) is coated with a super-hydrophobic layer where the super-hydrophobic material is comprised of manganese oxide polystyrene (MnO₂/PS), zinc oxide polystyrene (ZnO/PS), precipitated calcium carbonate, carbon nano-tube structures, or silica nano-coating. In the preferred embodiment, the internal and external aspects of the internal catheter device are coated with a silica-based super-hydrophobic layer.

In yet another embodiment, the urine collection device (6) is connected to an internal catheter device by at least one tube (5). Both the internal and external aspects of the internal catheter device are coated with a super-hydrophobic layer where the super-hydrophobic material is comprised of manganese oxide polystyrene (MnO₂/PS), zinc oxide polystyrene (ZnO/PS), precipitated calcium carbonate, carbon nano-tube structures, or silica nano-coating. In the preferred embodiment, the internal and external aspects of the internal catheter device are coated with a silica-based super-hydrophobic layer. The interior aspects of the tubes (5) are coated with a super-hydrophobic layer where the super-hydrophobic material is comprised of manganese oxide polystyrene (MnO₂/PS), zinc oxide polystyrene (ZnO/PS), precipitated calcium carbonate, carbon nano-tube structures, or silica nano-coating. In the preferred embodiment, the interior aspects of the tube (5) are coated with a silica-based super-hydrophobic layer. In yet another embodiment, the internal catheter device comprises methods as taught in U.S. Pat. No. 4,575,371, U.S. Pat. No. 6,090,075, U.S. Pat. No. 8,475,434 B2, U.S. Pat. No. 8,529,549 B2, U.S. Pat. No. 1,581,508 A, U.S. Pat. No. 3,154,080 A, U.S. Pat. No. 3,556,294 A, U.S. Pat. No. 3,648,704 A, U.S. Pat. No. 3,705,875 A, U.S. Pat. No. 4,062,363 A, U.S. Pat. No. 4,140,127 A, U.S. Pat. No. 4,235,232 A, U.S. Pat. No. 4,850,350 A, U.S. Pat. No. 5,041,085 A, U.S. Pat. No. 5,147,341, U.S. Pat. No. 5,167,646 A, each of which is hereby incorporated by reference in its entirety.

In the preferred embodiment, the internal aspect of the bladder (7), internal aspects of the tubes (5), internal aspects of the external catheter devices (12 and 23), and internal and external aspects of the internal catheter device, are coated with a super-hydrophobic layer where the super-hydrophobic material is comprised of manganese oxide polystyrene (MnO₂/PS), zinc oxide polystyrene (ZnO/PS), precipitated calcium carbonate, carbon nano-tube structures, or silica nano-coating. In the preferred embodiment, the internal and external aspects of the internal catheter device are coated with a silica-based super-hydrophobic layer.

In yet another embodiment, the urine collection device (6) is connected to a device that is attached to or inserted into a user's body for the purpose of fluid collection, including but not limited to urinary catheters, Foley catheters, Suprapubic catheters, nephrosotomy catheters, external catheters and external incontinence devices. These devices can be indwelling, such as Foley catheters, or attached externally to the user, such as the condom catheter and the external incontinence devices disclosed in U.S. Pat. No. 5,263,947, U.S. Pat. No. 5,827,247, and U.S. Pat. No. 5,830,932, each of which is hereby incorporated by reference in its entirety.

In one embodiment, the urine collection device (6), tube (5), and male external catheter device (12) are a disposable unit.

In the preferred embodiment, the urine collection device (6), tube (5), and male external catheter device (12) are a disposable unit comprising biodegradable plastic and rubber.

In one embodiment, the urine collection device (6), tube (5), and female external catheter device (23) are a disposable unit.

In the preferred embodiment, the urine collection device (6), tube (5), and female external catheter device (23) are a disposable unit comprising biodegradable plastic and rubber.

In one embodiment, the disposable urine collection device (6) is connected by a tube (5) to a long-term use internal catheter.

Figures are not to scale.

### PATENT CITATIONS

### CA 1284002 C

### EP 0,391,814 A

### EP 0,526,225 A1

### EP 0,693,915 B1

### EP 1,447,067 A1

### EP 1216673 B1

### EP 1633296 B2

U.S. Pat. No. 1,581,508 A
U.S. Pat. No. 1,687,012 A
U.S. Pat. No. 2,310,505 A
U.S. Pat. No. 2,389,831
U.S. Pat. No. 2,648,335 A
U.S. Pat. No. 2,887,109 A

### US 2005,0,177,133 A1

### US 2006,0,079,854 A1

### US 2011,0,028,982 A1

### US 20110137274 A1

### US 20130066289 A1

### US 20130138066 A1

U.S. Pat. No. 3,154,080 A
U.S. Pat. No. 3,282,412 A
U.S. Pat. No. 3,292,626 A
U.S. Pat. No. 3,295,145 A
U.S. Pat. No. 3,297,152 A
U.S. Pat. No. 3,403,682
U.S. Pat. No. 3,405,714 A
U.S. Pat. No. 3,520,305
U.S. Pat. No. 3,556,294 A
U.S. Pat. No. 3,559,651 A
U.S. Pat. No. 3,618,606 A
U.S. Pat. No. 3,648,704 A
U.S. Pat. No. 3,705,875 A
U.S. Pat. No. 3,739,783
U.S. Pat. No. 3,739,783
U.S. Pat. No. 3,780,739 A
U.S. Pat. No. 3,788,324
U.S. Pat. No. 3,822,704 A
U.S. Pat. No. 3,835,857
U.S. Pat. No. 3,863,638
U.S. Pat. No. 3,902,496 A
U.S. Pat. No. 4,062,363 A
U.S. Pat. No. 4,084,590 A
U.S. Pat. No. 4,140,127 A
U.S. Pat. No. 4,187,851
U.S. Pat. No. 4,187,851
U.S. Pat. No. 4,233,978 A
U.S. Pat. No. 4,235,232 A
U.S. Pat. No. 4,284,079
U.S. Pat. No. 4,339,485
U.S. Pat. No. 4,402,688
U.S. Pat. No. 4,484,917 A
U.S. Pat. No. 4,575,371
U.S. Pat. No. 4,850,350 A
U.S. Pat. No. 4,889,597 A
U.S. Pat. No. 4,935,022 A
U.S. Pat. No. 4,231,370 A
U.S. Pat. No. 5,041,085 A
U.S. Pat. No. 5,147,341
U.S. Pat. No. 5,167,646 A
U.S. Pat. No. 5,263,947
U.S. Pat. No. 5,263,947
U.S. Pat. No. 5,334,175
U.S. Pat. No. 5,827,247
U.S. Pat. No. 5,830,932
U.S. Pat. No. 5,342,861 A
U.S. Pat. No. 6,090,075
U.S. Pat. No. 6,428,521 B1
U.S. Pat. No. 6,075,178 A
U.S. Pat. No. 7,918,836 B2
U.S. Pat. No. 7,159,532 B2
U.S. Pat. No. 7,365,238 B2
U.S. Pat. No. 8,075,538 B2
U.S. Pat. No. 8,475,434 B2
U.S. Pat. No. 8,529,549 B2
U.S. Pat. No. 8,217,217 B2
U.S. Pat. No. 8,383,875 B2

### U.S. RE33206

### U.S. RE33206 E

### WO 2001041691 A1

WO 9,427,530

What is claimed is:

1. A urine collection assembly, comprising:
A male external catheter comprising;
a rollable condom-like tube with two strips of double-sided adhesive tape at its proximal two-thirds,
at least one drainage outlet at its distal end, and
A urine collection device comprising;
an absorbent pad enclosed in a backing sheet configured to be wearable in close proximity to a user's body, below the waist, pre-pubic, pubic, or perineal areas,
and at least one entrance conduit having an inlet to receive urine from the external catheter or an internal catheter.

2. The urine collection device according to claim 1, said urine collection device communicating with and receiving urine directly from an external or internal catheter without drainage tubes.

3. The urine collection device according to claim 1 comprising a bladder, tube, or other conduit means, connected to the entrance conduit at its proximal aspect with openings at its distal aspect to allow the distal aspect of the urine collection device to absorb urine prior to the proximal aspect.

4. The urine collection device according to claim 1 comprising an air release valve, said air valve activated upon air pressure to release air from the urine collection device.

5. The urine collection device according to claim 1 comprising a gauge, said gauge calibrated to indicate the volume of urine absorbed so it is known by a user what volume of urine has been absorbed and when the device if fully saturated with urine and should be replaced.

6. The urine collection device according to claim 1 comprising a urinalysis strip, said urinalysis strip indicating by qualitative means the chemistry of urine absorbed into the device.

7. The urine collection device according to claim 1 comprising a holster, said holster comprising a front elastic band and a cover elastic band, said front elastic band and cover elastic band causing the holster cover and front flap to exert steady pressure on said urine collection device to expel gas from said urine collection device via the air release valve.

8. The urine collection device according to claim 7 wherein said holster further comprises a bottom flap, a front flap, and a superior flap to allow for access through the inferior, superior, and frontal aspects of the holster for easy insertion and removal of the urine collection device so it can be easily used by persons with limited dexterity or muscle strength, when the device is configured for use below the waist but not at the pubic or perineal configurations.

9. The urine collection assembly according to claim 1 wherein said urine collection assembly is configured, with or without tubes connecting an internal or external catheter aspect to the urine collection aspect, to be worn as an always dry diaper.

10. The male external catheter according to claim 1 comprising a sealing membrane with a slit at its center to receive the penile shaft, said sealing membrane separating the proximal two-thirds of the catheter from the distal one-third.

11. The male external catheter according to claim 1 wherein the interior aspect of the distal one-third, the sealing membrane, and the drainage tube(s) are coated with a super-hydrophobic material, said super-hydrophobic material comprising manganese oxide polystyrene (MnO₂/PS), zinc oxide polystyrene (ZnO/PS), precipitated calcium carbonate, carbon nano-tube structures, or silica nano-coating.

12. The male external catheter according to claim 10 wherein said sealing membrane comprises two layers, said layers containing a slit in the center to receive the penile shaft.

13. The male external catheter according to claim 11 wherein said layers comprise a drying agent and charcoal.

14. The male external catheter according to claim 1 comprising a biodegradable material, said biodegradable material coated with a super-hydrophobic layer, said super-hydrophobic layer comprising of manganese oxide polystyrene (MnO₂/PS), zinc oxide polystyrene (ZnO/PS), precipitated calcium carbonate, carbon nano-tube structures, or silica nano-coating.

15. A urine collection assembly, comprising:
A female external catheter comprising;
a ridged receptacle, said receptacle communicating at its inferior aspect with at least one drainage outlet,
a spine propping said ridged receptacle,
a lip directing urine flow towards a urine collection device, and
A urine collection device comprising;
an absorbent pad enclosed in a backing sheet configured to be wearable in close proximity to a user's body, below the waist, pre-pubic, pubic, or perineal areas,
and at least one entrance conduit having an inlet to receive urine from the external catheter or an internal catheter.

16. The urine collection device according to claim 15, said urine collection device communicating with and receiving urine directly from said female external catheter or an internal catheter, without drainage tubes.

17. The urine collection assembly according to claim 15 wherein said urine collection assembly is configured, with or without tubes connecting an internal or external catheter aspect to the urine collection aspect, to be worn as an always dry diaper.

18. The female external catheter according to claim 15 wherein the receptacle and drainage outlets are coated with a super-hydrophobic layer, said super-hydrophobic layer comprising of manganese oxide polystyrene (MnO₂/PS), zinc oxide polystyrene (ZnO/PS), precipitated calcium carbonate, carbon nano-tube structures, or silica nano-coating.

19. The female external catheter according to claim 15 comprising a vaginal skin barrier, said vaginal skin barrier preventing splashing of urine and directing urine flow to the drainage outlet(s).

20. The female external catheter according to claim 15 comprising a biodegradable material, said biodegradable material coated with a super-hydrophobic layer, said super-hydrophobic layer comprising of manganese oxide polystyrene (MnO₂/PS), zinc oxide polystyrene (ZnO/PS), precipitated calcium carbonate, carbon nano-tube structures, or silica nano-coating.
